(19) United States
(12) Plant Patent Application Publication
(10) Pub. No.: US 2003/0188355 P1
(43) Pub. Date: Oct. 2, 2003

(54) Acer palmatum plant named 'Fujinami Nishiki'

(76) Inventor: Yoshimichi Hirose, Iwakuni (JP)

Correspondence Address: CHRISTIE, PARKER & HALE, LLP, 350 WEST COLORADO BOULEVARD, SUITE 500, PASADENA, CA, 91105, US

(21) Appl. No.: 10/107,904

(22) Filed: Mar. 26, 2002

(51) Int. Cl. A01H 5/00

(52) U.S. Cl. PLT/224

(57) ABSTRACT

A new plant variety of Acer palmatum characterized by Pink sectoral leaf variegation.

### BACKGROUND OF THE INVENTION

The present invention relates to a new and distinct variety of Acer palmatum Thunb., which was discovered as a branch sport of an unnamed variety of Acer palmatum (unpatented) in Kawaguchi City, Saitama, Japan, by the originator, Mr. Yoshimichi Hirose. The varietal denomination of the new variety is ‘FUJINAMI NISHIKI’.

The genus Acer is included in the family Aceraceae that comprises 2 genera of shrubs and trees native to northern temperate regions and tropical mountains. Acer comprises about 150 species of deciduous or evergreen trees and shrubs, native to North and Central America, Europe, North Africa and Asia.

Acer palmatum is a native of Japan and Korea. Acer palmatum generally has 5 to 7 lobed, palmate leaved, lobes that are deeply divided, lanceolate to oblong, biserrate, cuspidate. Petioles are approximately 4 cm long.

### SUMMARY OF THE INVENTION

The new variety was discovered as a branch sport and differs from other cultivars known to the inventor by its Pink sectoral leaf variegation. Asexual reproduction of the new variety by grafting has confirmed that the distinctive characteristics of the new variety are stable and transmitted to succeeding generations and that the new variety reproduces true to type.

### COMPARISON WITH PARENT

‘FUJINIMI NISHIKI’ is distinguished from its parent and all other varieties of Acer palmatum of which I am aware by its Pink sectoral leaf variegation. In contrast, the parent of ‘FUJINIMI NISHIKI’ exhibits all Red foliage.

### BRIEF DESCRIPTION OF ILLUSTRATION

The accompanying photographic illustration shows a plant of the new cultivar as true to color as is reasonably possible to make in an illustration of this character.

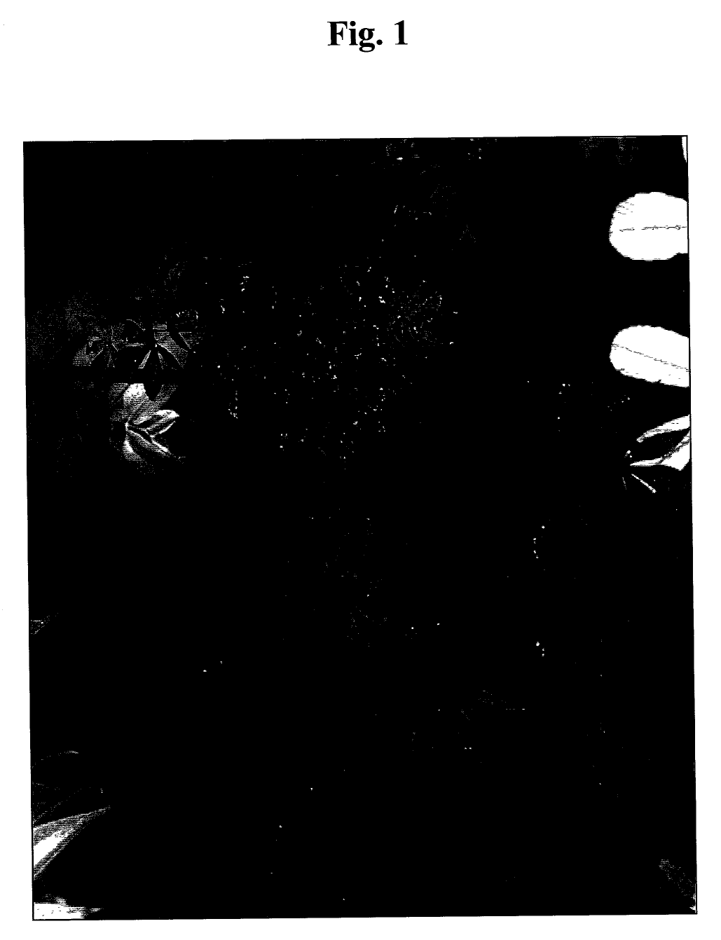

FIG. 1 illustrates the distinctive leaf variegation of the new variety.

### DESCRIPTION OF THE NEW VARIETY

ACER PALMATUM ‘FUJINIMI NISHIKI’ has not been observed under all possible environmental, cultural and light conditions. The following observations and descriptions are of 4 year-old plants grown in 5-gallon nursery containers in Forest Grove, Oreg., Lewisberry, Pa., and Kawaguchi City, Saitama, Japan. Phenotypic expression may vary with light intensity, cultural, and environmental conditions. In this description, color references are to The Royal Horticultural Society Colour Chart (2001) and terminology used in the color descriptions herein refers to plate numbers in this color chart. As with many plants in the Aceraceae family, phenotypic characteristics can be dramatically different in terms of growth morphology.

Classification:

Botanical.—Acer palmatum ‘FUJINIMI NISHIKI’ Thunb.

Parentage.—Branch sport of an unnamed, unpatented variety of Acer palmatum.

Propagation.—Grafting.

Plant:

Size.—8 to 10 meters tall at maturity.

Branching.—Typical Quantity Branches: More than 1000 Observed Quantity Branches: 15 Branch Size: Approximately 1 meter Branch Diameter: Base: 3 cm Apex: less than 1 cm Branch Coloration: Near Purple Group N77A

Habit.—Upright, oval.

Leaf:

Shape.—5 lobed, palmate.

Apex.—Acute.

Base.—Obtuse.

Margin.—Serrated.

Texture.—Moderately firm.

Substance.—Chartaceous.

Pattern of variegation.—Sectoral; most commonly 1 to 3 lobes of leaf entirely pinkish in color; occasional lobes in which irregularly alternating patters of ground color and variegation within or bounded by veins; or occasional leaves that are entirely of ground color or variegation color; in general, the percentage of the variegation portion of the leaf is 10% to 35%.

Leaf color.—Varies with intensity of sunlight and cultural conditions; the following color readings were taken in Forest Grove, Oreg. of a 4 year-old specimen grown in 5 gallon nursery containers. Upper side: Ground color near Violet Group 50A; variegation near Red Group 50A Underside: Ground color near Violet Group 50A; variegation near Red Group 50B Venation Upper Surface: Midvein depressed Under Surface: Midvein slightly raised Color: Variegated sectors near Red-Purple 71A; ground color sector near Red Group 50A Petioles Size: 3.5 cm long Color: Near Red Group 46A Internodes: 3 to 4 cm long Leaf size: From 4 to 7 cm long and 6 to 8 cm wide.

Hardiness.—USDA Zone 5 (20° to 10° F.).

Vigor.—Slow.

Pests/Diseases.—None noted.

INFLORESENCE None yet observed

REPRODUCTIVE ORGANS None yet observed

I claim:

1. A new plant variety of Acer palmatum of the variety substantially as shown and described.
